(19) United States
(12) Patent Application Publication
(10) Pub. No.: US 2010/0041900 A1
(43) Pub. Date: Feb. 18, 2010

(54) Isolation of lactam compound from adlay bran and its use on anti-proliferative cancer cells

(75) Inventors: Wen-Chang Chiang, Taipei City (TW); Yueh-Hsiung Kuo, Taipei City (TW); Ming-Yi Lee, Taipei City (TW)

Correspondence Address: BACON & THOMAS, PLLC, 625 SLATERS LANE, FOURTH FLOOR, ALEXANDRIA, VA, 22314-1176, US

(73) Assignee: KUANG TA FOODS CO., LTD, Tainan City (TW)

(21) Appl. No.: 12/222,608

(22) Filed: Aug. 13, 2008

(51) Int. Cl. C07D 491/107

(52) U.S. Cl. 548/410

(57) ABSTRACT

The invention relates to the active lactam compounds with inhibitory effect (anti-proliferative effect) on cancer cells, which are isolated from adlay (Coix lachryma-jobi L. var. ma-yuen Stapf) bran. In the present invention, structures and activities in vitro of the active lactam compounds are further characterized. The active compounds exhibited a strong anti-proliferative effect on cancer cells, such as human lung cancer cell and human colorectal carcinoma cell.

### FIELD OF THE INVENTION

The present relates to noval and known lactam compounds isolated from adlay (Coix lachryma-jobi L. var. ma-yuen Stapf) bran (AB), which compounds exhibit excellent anti-proliferative effect on cancer cells. The lactam compounds of the invention are useful as anti-proliferative agents, particularly may be used in combating cancers, such as lung cancer and colorectal carcinoma.

### BACKGROUND OF THE INVENTION

Adlay (Coix lachryma-jobi L. var. ma-yuen Stapf) is a grass crop that has long been used in traditional Chinese medicine and as a ‘nourishing’ food. Seed of adlay (so called as job's tears, or Chinese pearl barley) has Japanese name of ‘yokui-nin’. Adlay is mainly planted in Taiwan, China, and Japan, where it is considered a healthy food supplement. According to the ancient Chinese medical book Pen-Tsao-Kang-Mu (1596), the seed of adlay was used in China for the treatment of warts, chapped skin, rheumatism, and neuralgia, and as an anti-inflammatory or antihelmintic agent. Numerous recent reports have indicated that the consumption of adlay seed is beneficial to the human body (see, for, example, Hidaka et al., Biotherapy 5, 201-203, 1992; Huang et al., Food Sci. 26, 121-130, 1999; Tsai et al., Food Sci. 26, 265-276, 1999; Chiang et al., J. Agric. Food Chem. 48, 829-832, 2000; and Hsu et al., J. Agric. Food Chem. 51, 3763-3769, 2003).

In the past, a few scientific studies have identified active components in adlay. Kuo et al. (J. Agric. Food Chem. 50, 5850-5855, 2002) used DPPH-directed fractionation and identified six phenolic compounds including coniferyl alcohol, syringic acid, ferulic acid, syringaresinol, 4-ketopinoresinol, and a lignan known as mayuenolide. All compounds isolated from adlay hulls showed strong free radical scavenging activity. Nagao et al. (Phytochemistry 24, 2959-2962, 1985) isolated benzoxazinones from adlay seeds that showed anti-inflammatory activity. Takahashi et al. (Planta Med. 52, 64-65, 1986) reported that coixans A, B, and C isolated from adlay seeds have hypoglycemic activity in rats. Coixenolide was isolated from adlay seeds, and it exhibited antitumor activity towards Ehrlich ascites sarcoma in mice (Tanimura, Chem. Pharm. Bull. 9, 47-53, 1961; and Ukita and Tanimura, Chem. Pharm. Bull. 9, 43-46, 1961). The anti-tumor constituents of adlay include a-monolinolein (Tokuda et al., Planta Med. 56, 653-654, 1990), and free fatty acids such as palmitic, stearic, oleic, and linoleic acids (Numata et al., Planta Med. 60, 356-359, 1994).

In addition, recent studies indicate that adlay has anti-tumor effects. For example, Chiang et al. (J. Health Sci. 2, 113-122, 2000) found that adlay inhibited sarcoma-180 tumor in mice. Kuo et al. (J. Agric. Food Chem. 49, 1564-1570, 2001) indicated that a methanolic extract of adlay hull has anti-proliferative activity against human histolytic lymphoma U937 monocytic cells via apoptosis. Chang et al. (J. Agric. Food Chem. 51, 3656-3660, 2003) reported that a methanolic extract of adlay has an anti-proliferative effect on A549 lung cancer cells by inducing cell cycle arrest and apoptosis. Feeding mice a diet containing adlay reduced the number of surface lung tumors in mice. Shih et al. (Food Chem. Toxicol. 42, 1339-1347, 2004) showed that dehulled adlay suppressed early events in colon carcinogenesis and it also reduced COX-2 protein expression.

Recent studies have shown that adlay bran may inhibit cancer cell growth, but only a few anti-tumor specific compounds have been identified. In the present invention, adlay bran is extracted with specific organic solvents, and compounds that inhibit the proliferation of lung and colon cancer cells are isolated from the extracts.

### SUMMARY OF THE INVENTION

In one aspect, the present invention provides a lactam compound isolated from the methanolic extracts of adlay bran (AB), which exhibits anti-proliferative effect on cancer cells. The purified lactams with anti-cancer effect include three new lactam compounds that are previously undocumented: coixspirolactam A (1), coixspirolactam B (2), and coixspirolactam C (3); one isolated from the natural plant for the first time, coixlactam (4); and one known compound, methyl dioxindole-3-acetate (5).

In another aspect, the present invention provides anti-proliferative lactam compounds isolated from adlay bran, which comprise coixspirolactam A (1), coixspirolactam B (2), coixspirolactam C (3), coixlactam (4), and methyl dioxindole-3-acetate (5). In one embodiment of the invention, the lactam compounds effectively inhibit the proliferation of lung and/or colon cancer cells.

According to one object of the invention, a process for isolating anti-proliferative lactam compounds from adlay bran is provided. The process comprises the steps of that: the adlay bran obtained from dried and dehulled adlay seed is blended into powder form; the adlay bran powder is treated with alcohol, such as methanol to obtain methanol extract (ABM); the water suspension of dried ABM is partitioned with n-hexane, ethyl acetate, and 1-butanol to yield four subfractions named: ABM-Hex, ABM-EtOAc, ABM-BuOH, and ABM-H₂O; the ABM-EtOAc subfraction is subjected to column chromatography; and the fractions which show greater inhibition of cancer cell proliferation are combined and further purified by HPLC to yield the anti-proliferative lactam compounds. In one embodiment of the invention, the isolated anti-proliferative lactam compounds are: coixspirolactam A (1), coixspirolactam B (2), coixspirolactam C (3), and coixlactam (4), and methyl dioxindole-3-acetate (5).

### DETAILED DESCRIPTION OF THE INVENTION

The invention is further defined by reference to the following examples describing in detail the preparation of compounds of the invention. The following examples are set forth to assist in understanding the invention and should not be construed as specifically limiting the invention described and claimed herein. Such and other variations or modifications of the invention are to be considered to fall within the scope of the invention incorporated herein.

### EXAMPLES

### Example 1

### Isolation of Anti-Proliferative Lactam Compounds from Adlay Bran (AB)

After the harvest, the seeds of adlay were dried at ambient temperature with ventilation and dehulled by a grinder. The samples were divided into hull, testa, and dehulled. The dehulled adlay was separated into bran and polished adlay. Thereafter, adlay bran powder (20 kg) was extracted with methanol at room temperature for 5 day (3 times, each time 200 L) with continuous stirring. The plant residue was filtered off, and the methanolic extracts were combined and concentrated under reduced pressure by a rotary vacuum evaporator. The methanolic extracts of adlay bran were named ABM.

The dry methanolic extract (ABM, 1864 g) was suspended in 18.6 L of water, followed by partitioning with n-hexane (1 times, 18.6 L), ethyl acetate (3 times, each time 18.6 L), and 1-butanol (3 times, each time 18.6 L), yielding four subfractions named: ABM-Hex (n-hexane soluble fraction), ABM-EtOAc (ethyl acetate soluble fraction), ABM-BuOH (1-butanol soluble fraction), and ABM-H₂O (water soluble fraction). ABM-EtOAc (380 g) was coated with 380 g silica gel (230˜400 mesh), and then subjected to column chromatography on silica gel (230˜400 mesh) with successive elution by a Hex/EtOAc and EtOAc/MeOH gradient. Subfractions with the same TLC pattern were combined into one fraction, thus fifteen fractions were obtained. The fractions which showed greater inhibition of cancer cell proliferation were chromatographed with 20-40% Hex/EtOAc on a silica gel column using a CHCl₂/EtOAc gradient system to yield subfractions.

The subfractions were further purified by HPLC on a Lichrosorb Si-60 column at 3 mL/min, using 30 or 50 or 65 or 80% EtOAc/CHCl₂ as the eluent to yield five compounds, coixspirolactams A (1) (12.3 mg), B (2) (7.8 mg), C (3) (7.5 mg), and coixlactams (4) (9.1 mg), methyl dioxindole-3-acetate (5) (11.3 mg). The structures of the five compounds are represented by following formula (1), (2), (3), (4), and (5), respectively.

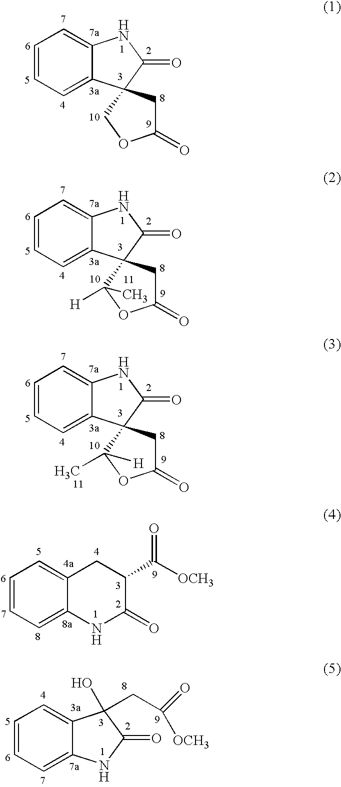

### Example 2

### Characterization of the Purified Anti-Proliferative Lactam Compounds

The compound (1) purified by the process described in Example 1 was characterized by following properties:

pale yellow oil. [α]_{D}²⁵=+9.7 (c=0.70, MeOH). UV (MeOH) λₘₐₓ nm (log ε): 250 (3.72). EIMS m/z (%): 203 (M⁺, 8), 167 (5), 146 (2), 145 (22), 137 (100), 117 (8), 107 (7), 84 (14). HREIMS m/z: 203.0580, calcd 203.0582 for C₁₁H₉NO₃. IR νₘₐₓ (film)/cm⁻¹: 3289, 1785, 1730, 1619, 1505, 1367, 1178. ¹H and ¹³C NMR data are presented in Table 1.

The compound (2) purified by the process described in Example 1 was characterized by following properties:

pale yellow oil. [α]_{D}²⁵=−4.3 (c=0.40, MeOH). UV (MeOH) λₘₐₓ nm (log ε): 251 (3.78). EIMS m/z (%): 217 (M⁺, 9), 174 (3), 173 (26), 146 (8), 145 (100), 144 (5), 117 (34), 107 (5), 90 (9). HREIMS m/z: 217.0738, calcd 217.0739 for C₁₂H₁₁NO₃. IR νₘₐₓ (film)/cm⁻¹: 3268, 1789, 1726, 1621, 1521, 1360, 1189. ¹H and ¹³C NMR data are presented in Table 1.

The compound (3) purified by the process described in Example 1 was characterized by following properties:

pale yellow oil. [α]_{D}²⁵=5.9 (c=0.39, MeOH). UV (MeOH) λₘₐₓ nm (log ε): 252 (3.61). EIMS m/z (%): 217 (M⁺, 9), 174 (3), 173 (26), 146 (8), 145 (100), 144 (5), 117 (34), 107 (5), 90 (9). HREIMS m/z: 217.0738, calcd 217.0739 for C₁₂H₁₁NO₃. IR νₘₐₓ (film)/cm⁻¹: 3269, 1795, 1736, 1625, 1471, 1200. ¹H and ¹³C NMR data are presented in Table 1.

The compound (4) purified by the process described in Example 1 was characterized by following properties:

pale yellow oil. [α]_{D}²⁵=+5.1 (c=0.21, MeOH). UV (MeOH) λₘₐₓ nm (log ε): 248 (3.81). EIMS: m/z (%) 205 (M⁺, 46), 173 (18), 146 (33), 145 (100), 128 (10), 117 (28). HREIMS m/z: 205.0736, calcd 205.0739 for C₁₁H₁₁NO₃. IR νₘₐₓ (film)/cm⁻¹: 3376, 1732, 1690, 1626, 1490, 1228, 1062. ¹H and ¹³C NMR data are presented in Table 1. The synthesis of compound (4) is described by Crotti et al. (J. Health Sci. 2, 113-122, 1986), but in this invention it was isolated from a natural source for the first time.

The compound (5) purified by the process described in Example 1 was characterized by following properties:

pale yellow oil. [α]_{D}²⁵=−1.4 (c=0.45, MeOH). UV (MeOH) λₘₐₓ nm (log ε): 252 (3.25). EIMS m/z (%): 276 (M⁺, 5), 261 (12), 232 (14), 220 (55), 205 (100), 177 (36), 149 (15), 91 (14), 57 (67). HREIMS m/z: 276.1735 calcd 276.1725 for C₁₇H₂₄O₃. IR νₘₐₓ (film)/cm⁻¹: 3337, 1725, 1626, 1202. ¹H NMR (CDCl₃, 500 MHz) δ8.08 (br s, 1H, N—H), 7.36 (d, J=7.5 Hz, 1H, H-4), 7.26 (t, J=7.5 Hz, 1H, H-6), 7.05 (t, J=7.5 Hz, 1H, H-5), 6.86 (d, J=7.5 Hz, 1H, H-7), 3.66 (s, 3H, OCH₃), 2.94, 2.92 (d, J=16.6 Hz, each 1H, H-8). ¹³C NMR (CDCl₃, 125 MHz) δ177.86 (C-2), 170.93 (C-9), 140.41 (C-7a), 130.21 (C-3a), 129.59 (C-4), 124.30 (C-6), 123.25 (C-5), 110.38 (C-7), 73.70 (C-3), 52.16 (—OCH₃), 40.78 (C-8). The spectra data of compound 5 were in agreement with the reported literature values (Taichi et al., Chem. Pharm. Bull. 36, 578-581, 1988).

TABLE 1
¹³C and ¹H NMR spectral data (500 and 125 MHz, in CDCl₃, J
in Hz) for Compounds 1, 2, and 3.

| | 1 | | 2 | | 3 | |
|---|---|---|---|---|---|---|
| no | δ_{C} | δ_{H} | δ_{C} | δ_{H} | δ_{C} | δ_{H} |
| 1 | — | 8.62 br s | — | 8.90 br s | — | 8.90 br s |
| 2 | 174.3 | — | 173.5 | — | 173.8 | — |
| 3 | 51.0 | — | 54.5 | — | 55.8 | — |
| 3a | 129.6 | — | 141.0 | — | 139.8 | — |
| 4 | 122.7 | 7.30 d (7.6) | 110.6 | 7.29 d (8.0) | 110.5 | 7.30 d (7.8) |
| 5 | 130.9 | 7.23 t (7.6) | 123.3 | 7.25 t (8.0) | 124.3 | 7.09 t (7.8) |
| 6 | 123.7 | 7.09 t (7.6) | 129.6 | 7.09 t (8.0) | 129.5 | 7.20 t (7.8) |
| 7 | 110.6 | 6.93 d (7.6) | 122.9 | 6.96 d (8.0) | 123.4 | 6.95 d (7.8) |
| 7a | 139.9 | — | 127.5 | — | 128.2 | — |
| 8 | 38.6 | 3.13 d (17.5),ₑₓₒ | 38.6 | 2.97 d (17.5),ₑₓₒ | 40.2 | 3.30 d (17.2),ₑₓₒ |
| | | 2.72 d (17.5),_{endo} | | 2.94 d (17.5),_{endo} | | 2.68 d (17.2),_{endo} |
| 9 | 177.8 | — | 177.9 | — | 177.9 | — |
| 10 | 74.7 | 4.57 d (9.0),ₑₓₒ | 82.1 | 4.73 q (6.4),_{endo} | 81.5 | 4.86 q (6.8),ₑₓₒ |
| | | 4.37 d (9.0),_{endo} | | | | |
| 11 | — | — | 14.9 | 1.34 d (6.4),ₑₓₒ | 15.4 | 1.09 d (6.8),_{endo} |

### Example 3

### Anti-Proliferative Effect of the Isolated Lactam Compounds—MTT Assay

The cell lines A549 (human lung cancer), HT-29 (human colon carcinoma), and COLO 205 (human colon carcinoma) were obtained from the American Type Culture Collection (ATCC) (Rockville, Md.). These cell lines were maintained in DMEM containing 10% heat-inactivated fetal bovine serum, 100 units/mL of penicillin and 100 μg/mL of streptomycin, and were kept at 37° C. in a 5% CO₂ incubator.

MTT assay was performed as described by Hansen et al. (Hansen et al., J. Immunol. Methods 119, 203-210, 1989). A549 (1.5×10³ cells/well), HT-29 (2×10³ cells/well), and COLO 205 (3×10³ cells/well) were seeded in 96-well plates and grown overnight. Cells were then incubated in 10% FBS medium containing different amounts of adlay bran subfractions for 48 hours. At the end of incubation, media were replaced with 100 μL of reagent (MTT, 1 mg/mL) and incubated in a 5% CO₂ incubator at 37° C. for another 2 h. The medium was then discarded and 100 μL of DMSO was added to each well. The absorbance was measured at 570 nm by scanning with a micro-plate reader (Molecular Devices, Calif.). Each sample was analyzed six times, and the values were averaged. For the determination of IC₅₀ (the concentration of each substance required to inhibit cell proliferation by 50%), each sample was measured at five different concentrations by the MTT test. The IC₅₀ was obtained by interpolation from linear regression analysis (showed in Table 2). The IC₅₀ values of each sample were collected from three replicates, and then mean±standard deviation values were obtained for the results of the IC₅₀ assay.

TABLE 2
Growth inhibition of adlay bran extracts and the compounds
isolated from ABM-EtOAc on A549, HT-29, and COLO 205
cells.

| | IC₅₀^{a} (μg/mL) | | |
|---|---|---|---|
| Samples | A549 | HT-29 | COLO 205 |
| ABM-Hex | 338.40 ± 29.85 | >500^{b} | 422.17 ± 37.79 |
| ABM-EtOAc | 81.68 ± 8.26 | 131.33 ± 15.97 | 153.18 ± 22.03 |
| 1 | 37.10 ± 1.93 | 34.43 ± 3.53 | 35.94 ± 4.65 |
| 2 | 28.57 ± 2.04 | 32.70 ± 2.29 | 41.29 ± 3.52 |
| 3 | 31.75 ± 2.58 | 37.55 ± 3.08 | 48.12 ± 4.15 |
| 4 | 65.00 ± 3.32 | 52.63 ± 2.64 | 72.57 ± 5.31 |
| 5 | 42.63 ± 2.31 | 48.94 ± 3.83 | 45.51 ± 6.42 |
| ABM-BuOH | 370.64 ± 27.63 | >500 | >500 |
| ABM-H₂O | >500 | >500 | >500 |

^{a}IC₅₀ is the concentration required for 50% growth inhibition of cancer cells.
^{b}Compounds having IC₅₀ value >500 μg/mL are not included in the table.

As showed in Table 2, the IC₅₀ values of compounds (1), (2), (3), (4), and (5) against human lung cancer cell A549, human colorectal carcinoma cell HT-29, and COLO 205 cells were 28.6-72.6 μg/mL. These five purified compounds prevented the proliferation of A549, HT-29 and COLO 205 cells in the following orders of potency 2>3>1>5>4, 2>1>3>5>4, and 1>2>3>5>4, respectively.

From the results as described above, the present invention discloses the anti-proliferative effects of five lactam compounds (compound 1-5) obtained from methanol extract of adlay bran for the first time. In these compounds, including three new compounds; coixspirolactam A (1), coixspirolactam B (2), and coixspirolactam C (3); one further compound that has been isolated from the natural plant coixlactam (4) for the first time; and one known compound methyl dioxindole-3-acetate (5). By the results of growth-inhibiting test on human lung cancer cell A549, human colorectal carcinoma cell HT-29, and COLO 205 cells, it shows that all of these five lactam compounds inhibit human cancer cells. Thus, the lactam compounds of the invention may be potential candidates for anti-proliferative agents of cancers, and further useful in preparing medicines for preventing and/or treating cancers.

1. A lactam compound, which is represented by formula (1).

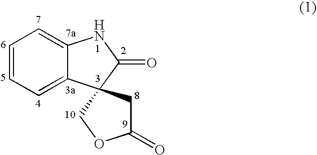

2. A lactam compound, which is represented by formula (2).

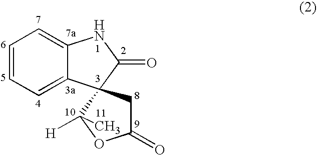

3. A lactam compound, which is represented by formula (3).

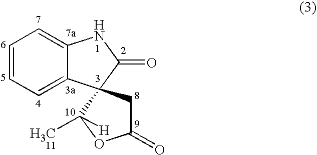

4. The lactam compound of claim 1, 2, or 3, which is isolated from adlay (Coix lachryma-jobi L. var. ma-yuen Stapf) bran.

5. The lactam compound of claim 1, 2, or 3, which exhibits anti-proliferative effect on cancer cells.

6. A process for isolating anti-proliferative lactam compounds from adlay bran, which comprises the steps of:
(1) obtaining adlay bran from dried and dehulled adlay seed, and blending it into powder form;
(2) treating the adlay bran powder with alcohol to obtain alcohol extract;
(3) extracting the water suspension of the dried alcohol extract with n-hexane, ethyl acetate, and 1-butanol to yield four subfractions: ABM-Hex, ABM-EtOAc, ABM-BuOH, and ABM-H₂O;
(4) subjecting the ABM-EtOAc subfraction to column chromatography, and collecting the fractions which show greater inhibition of cancer cell proliferation; and
(5) purifying the anti-proliferative subfractions by HPLC to yield the anti-proliferative lactam compound of formula (1), (2), or (3) defined in claim 1, 2, or 3.

7. The process of claim 6, wherein the alcohol is methanol or ethanol.
